(19) United States
(12) Patent Application Publication
(10) Pub. No.: US 2025/0387437 A1
(43) Pub. Date: Dec. 25, 2025

(54) PHARMACEUTICAL COMPOSITION FOR PREVENTING OR TREATING ALOPECIA INCLUDING AKKERMANSIA

(71) Applicant: ENTEROBIOME INC., Goyang-si, Gyeonggi-do (KR)

(72) Inventor: Jae-Gu SEO, Gimpo-si (KR)

(73) Assignee: ENTEROBIOME INC., Goyang-si, Gyeonggi-do (KR)

(21) Appl. No.: 18/879,100

(22) PCT Filed: Apr. 14, 2023
(86) PCT No.: PCT/KR2023/005123

§ 371 Date: Dec. 26, 2024

(30) Foreign Application Priority Data
Jul. 14, 2022 (KR) 10-2022-0086771

(51) Int. Cl. A61K 35/74; A61K 8/99; A61K 31/506; A61K 31/519; A61K 31/567; A61K 31/58; A61P 17/14; A61Q 7/00

(52) U.S. Cl. CPC A61K 35/74; A61K 8/99; A61K 31/506; A61K 31/519; A61K 31/567; A61K 31/58; A61P 17/14; A61Q 7/00

(57) ABSTRACT

A pharmaceutical composition, a food composition, and a cosmetic composition for preventing or treating alopecia, and their uses are disclosed. The compositions contain, as an active ingredient, an Akkermansia sp. strain, vesicles of the strain, a proteome and metabolome of the strain, a culture of the strain, a lysate of the strain, an extract of the strain, an extract of the culture, or an extract of the lysate. The compositions for preventing or treating alopecia are safe for the human body, have no side effects, and have excellent effects of combating alopecia and promoting hair growth.

### TECHNICAL FIELD

The present invention relates to a pharmaceutical composition for preventing or treating alopecia, and more specifically, to a pharmaceutical composition for preventing or treating alopecia including bacteria of the genus Akkermansia as an active ingredient, and a food composition and cosmetic composition for preventing or improving alopecia or promoting hair growth.

### BACKGROUND ART

With the development of modern society, the number of alopecia patients has been increasing rapidly, and the age group of patients who suffer from alopecia is gradually decreasing, and the number of patients suffering from alopecia among those in their 20's and 30's is increasing rapidly.

It is reported that people with more severe alopecia are more likely to experience psychological depression and anxiety, perceive high levels of stress, are faced with difficulties in adapting interpersonal or heterosexual relationships with the opposite gender, and fail to maintain good family relationships. Thus, alopecia has become a major concern in health science.

Although the exact cause of alopecia is not clearly known yet, it is reported that alopecia is possibly caused by genetic factors, hormonal abnormalities, immunological abnormalities, and the like.

Androgenetic alopecia (AGA) is alopecia in which male hormone acts on male hormone-sensitive hair follicles to form vellus hair, and it is guessed that the genetic predisposition is largely related. Female pattern alopecia is alopecia that is thought to be caused by a decrease in the amount of estrogen relative to the amount of androgen in the bloodstream. It often occurs after the menopause, and female pattern alopecia might be improved by hormone replacement therapy but is intractable in many cases.

Drugs that can be used to AGA include minoxidil, male hormone activity inhibitors such as finasteride, female hormone drugs such as estrogen, estradiol, and progesterone, antifungal drugs such as ketoconazole, pentadecane, cytopurine (6-benzylaminopurine), t-flavanone, adenosine, and the like.

However, minoxidil has side effects such as drug-induced contact alopecia, hair hyperplasia, a decrease in blood pressure, and a decrease in heart rate, and there is the problem that when its use is stopped, the symptoms return. Meanwhile, finasteride has side effects such as prostate hyperplasia, erectile dysfunction, and ejaculatory disorder, and there is a side effect that the symptoms worsen when its use is stopped. In addition, minoxidil may have the risk of hirsutism or birth defects when used in a high dose in women, and finasteride has the risk of birth defects, so that administration to women, particularly, pregnant women is prohibited.

In addition, there are various hair loss products containing ingredients for promoting blood circulation, enhancing hair root function, moisturizing the scalp, combating dandruff, preventing oxidation, extending the anagen, etc. as active ingredients, but such existing formulations have rarely been proven to have any obvious effects, and have side effects.

Meanwhile, in an effort to obtain the effect of combating alopecia by using microorganisms, Korean Patent No. 1600012 discloses a therapeutic agent for alopecia in which Zizania latifolia Turcz, and malt are fermented by using microorganisms such as Lactobacillus plantarum and raw materials such as Houttuynia cordata and Polygonum multiflorum are then added thereto. Korean Patent Publication No. 2022-0021144 discloses a pharmaceutical composition for treating benign prostatic hyperplasia or alopecia comprising lactic acid bacteria of the Enterococcus genus as an active ingredient. However, none of these compositions has yet shown a significant effect in the treatment of alopecia.

### DISCLOSURE OF INVENTION

### Technical Problem

To solve the above problems, one object of the present invention is to provide a pharmaceutical composition for preventing or treating alopecia, the pharmaceutical composition being safe for the human body, having no side effects, and having excellent effects of combating alopecia and promoting hair growth or hair restoration.

Another object of the present invention is to provide a food composition for preventing or ameliorating alopecia comprising live bacteria, killed bacteria, or a mixture thereof of the genus Akkermansia as an active ingredient.

Still another object of the present invention is to provide a cosmetic composition capable of preventing, relieving, and combating scalp aging, thereby preventing, relieving, and ameliorating alopecia and promoting hair growth.

### Solution to Problem

In order to achieve the above-described objects of the present invention, the present invention provides a pharmaceutical composition for preventing or treating alopecia, the pharmaceutical composition comprising, as an active ingredient, an Akkermansia sp. strain, vesicles of the strain, a proteome and metabolome of the strain, a culture of the strain, a lysate of the strain, an extract of the strain, an extract of the culture, or an extract of the lysate and at least one pharmaceutically acceptable excipient.

The Akkermansia sp. strain may be an Akkermansia muciniphila EB-AMDK19 (KCTC 13761BP) strain. The Akkermansia sp. strain may be live bacteria, killed bacteria, or a mixture thereof.

The pharmaceutical composition for preventing or treating alopecia of the present invention may be used for the treatment of one or more alopecia selected from the group consisting of androgenetic alopecia, telogen effluvium, alopecia areata, alopecia totalis, hypotrichosis, hereditary hypotrichosis simplex, frontal fibrosing alopecia, cicatricial alopecia, lichen planophilaris, ring alopecia, scarring alopecia, nonscarring alopecia, chemotherapy induced alopecia, and alopecia universalis.

The pharmaceutical composition for preventing or treating alopecia of the present invention may further include at least one therapeutic agent for alopecia selected from the group consisting of finasteride, dutasteride, minoxidil, episteride, alfatradiol, tofacitinib, and ruxolitinib in addition to the Akkermansia sp. strain.

Another aspect of the present invention relates to a food composition for preventing or ameliorating alopecia, the food composition comprising, as an active ingredient, an Akkermansia sp. strain, vesicles of the strain, a proteome and metabolome of the strain, a culture of the strain, a lysate of the strain, an extract of the strain, an extract of the culture, or an extract of the lysate and at least one physiologically acceptable excipient, diluent or carrier.

Still another aspect of the present invention relates to a cosmetic composition for combating alopecia or promoting hair growth, the cosmetic composition comprising, as an active ingredient, an Akkermansia sp. strain, vesicles of the strain, a proteome and metabolome of the strain, a culture of the strain, a lysate of the strain, an extract of the strain, an extract of the culture, or an extract of the lysate and at least one cosmetically acceptable excipient, diluent or carrier.

### Advantageous Effects of Invention

According to various embodiments of the present invention, it is possible to provide a pharmaceutical composition for preventing or treating alopecia, the pharmaceutical composition having excellent effects of treating alopecia and promoting hair growth but having few side effects.

The pharmaceutical composition for preventing or treating alopecia of the present invention has an effect of preventing and combating a phenomenon that the hair falls off from the scalp or a phenomenon that the hair is thinned or tapered.

The food composition of the present invention has an effect of implementing not only a hair growth function which generates new hair, or a function for promoting the hair growth, but also a function of promoting the delay from anagen to catagen of the hair cycle and enabling existing hair to grow healthy.

The cosmetic composition of the present invention comprising live bacteria, killed bacteria, inactivated or a pasteurized mixture thereof of the genus Akkermansia can prevent, relieve, and combat scalp aging, thus can improve the effect of combating alopecia, and can provide a significant effect of promoting hair growth or hair restoration.

### BRIEF DESCRIPTION OF DRAWINGS

FIG. 1 shows photographs showing regrowth of hair as the number of days of sample application has elapsed when each sample is administered to an alopecia mouse model in Example of the present invention.

FIGS. 2a-b are graphs showing the results of hair length change in each experimental group.

FIGS. 3a-c are graphs showing the measurement results of the body weight, liver tissue weight, and prostate tissue weight after completion of the experiment at week 5 in each experimental group.

FIG. 4 shows photographs obtained by photographing prostate after completion of the experiment at week 5 in each experimental group.

FIGS. 5a-b are photographs of a side surface (FIG. 5a) and a front surface (FIG. 5b) observed through an optical microscope by staining, with hematoxylin-eosin, a skin tissue collected after the experiment at week 5 in each experimental group.

### MODE FOR THE INVENTION

Hereinafter, embodiments of the present invention will be described in detail. Detailed descriptions related to well-known functions or configurations may be omitted in order not to unnecessarily obscure subject matters of the present invention.

Throughout this specification and the claims, unless otherwise stated, the term “comprise or comprising” means that it includes an object, a step, or a group of objects and steps mentioned, and is not used to exclude any other object, step, or group of objects or steps.

As used herein, the terms such as “treat” and “treatment” mean that symptoms are temporarily or permanently relieved, the cause of the symptoms is removed, or the development of symptoms of a disease or condition is combated or delayed.

As used herein, the term “prevention” refers to all actions that inhibit alopecia or delay the onset of alopecia by the administration of the pharmaceutical composition according to the present invention.

As used herein, the term “amelioration” refers to all actions that reduce a parameter related to an abnormal state, for example, the degree of symptoms.

As used herein, the term “pharmaceutically acceptable” refers to a composition that is suitable for use in contact with tissue of a subject (e.g., human) because the benefit/risk ratio is reasonable without excessive toxicity, irritation, allergic reaction or other problems or complications, and is within the scope of sound medical judgment.

As used herein, the term “alopecia” refers to a phenomenon in which hair falls off from the scalp or a phenomenon in which the hair is thinned or tapered. The alopecia in the present invention may include androgenetic alopecia, telogen effluvium, anagen effluvium, alopecia areata, drug-induced telogen effluvium, postpartum telogen effluvium, etc. In addition, it may include all kinds of alopecia that form an abnormal hair loss pattern such as becoming vellus hair of the terminal hair, becoming a single hair of the group hair, and atrophy of the hair follicle due to a series of factors.

As used herein, the term “promotion of hair restoration” refers to a hair growth function of generating new hair, or a function of promoting hair growth, as well as a function of promoting the delay from anagen to catagen and enabling existing hair to grow healthy.

One aspect of the present invention relates to a pharmaceutical composition for preventing or treating alopecia, the pharmaceutical composition comprising, as an active ingredient, an Akkermansia sp. strain, vesicles of the strain, a proteome and metabolome of the strain, a culture of the strain, a lysate of the strain, an extract of the strain, an extract of the culture, or an extract of the lysate.

Any strain of the genus Akkermansia may be used as the Akkermansia sp. strain as long as it is commonly known, and for example, an Akkermansia muciniphila strain may be used. In the present invention, examples of the Akkermansia sp. strain include the Akkermansia muciniphila EB-AMDK19 strain, which are isolated and deposited by the present applicant. Specifically, under Budapest Treaty, it is preferable to use the Akkermansia muciniphila EB-AMDK19 strain deposited in the Korean Collection for Type Culture (KCTC) at the Korea Research Institute of Bioscience and Biotechnology on Dec. 5, 2018, and received an accession number of KCTC 13761BP.

The Akkermansia sp. strain may be viable (live), dormant, inactivated, pasteurized or killed or any combination thereof. As used above, the term “killed bacteria” refers to a strain inactivated by heat treatment. In one embodiment, the killed bacteria of the Akkermansia sp. strain of the present invention is preferably prepared by heat-treating the Akkermansia sp. strain at 50° C. to 100° C., preferably 60° C. to 95° C., and more preferably 70° C. to 90° C. for 5 minutes to 1 hour, but is not limited thereto. When the heat treatment temperature increases, the heat treatment time may be shortened.

The pharmaceutical composition for preventing or treating alopecia of the present invention may include live bacteria, killed bacteria, or a mixture thereof of the Akkermansia muciniphila in an amount ranging from about 1×10² CFU to about 1×10¹⁵ CFU, preferably from about 1×10⁴ CFU to about 1×10¹² CFU, more preferably from about 1×10⁵ CFU to about 1×10¹⁰ CFU, and even more preferably from about 1×10⁶ CFU to about 1×10⁹ CFU. When the amount of strain is less than the above range, it is difficult to exhibit sufficient effects of combating alopecia and promoting hair growth, and when the amount of strain is greater than the above range, it is difficult to expect an additional increase in effect.

In the present invention, live bacteria, killed bacteria, or a mixture thereof of the genus Akkermansia is substantially purified. The substantially purified Akkermansia live bacteria, killed bacteria or a mixture thereof may be included in the sample in an amount of at least about 50%, preferably about 60%, about 70%, about 80%, about 90%, about 95%, about 99% or more.

The pharmaceutical composition of the present invention may be used for the treatment of one or more alopecia selected from consist of androgenetic alopecia, telogen effluvium, alopecia areata, alopecia totalis, hypotrichosis, hereditary hypotrichosis simplex, frontal fibrosing alopecia, cicatricial alopecia, lichen planophilaris, ring alopecia, scarring alopecia, nonscarring alopecia, chemotherapy induced alopecia, and alopecia universalis.

The pharmaceutical composition of the present invention may further include at least one therapeutic agent for alopecia selected from the group consisting of finasteride, dutasteride, minoxidil, episteride, alfatradiol, tofacitinib, and ruxolitinib. The Akkermansia sp. strain of the present invention may be administered simultaneously with a therapeutic agent for alopecia as a single formulation, or may be administered simultaneously or sequentially with separate formulations. The pharmaceutical composition for preventing or treating alopecia of the present invention not only exhibits an excellent effect of combating alopecia when administered alone, but when administered in combination with a therapeutic agent for alopecia such as minoxidil or finasteride, the pharmaceutical composition also has a far better therapeutic effect than when the therapeutic agent for alopecia is administered alone since side effects of the therapeutic agent for alopecia are reduced and efficacy thereof is more activated.

In an embodiment of the present invention, the culture of the Akkermansia sp. strain refers to a culture product, such as a culture medium or a culture solution, containing some or all of the substances contained in the culture medium in which the strain was cultured, and a concentrate thereof. For example, it may refer to a substance including a metabolite or a secreted product resulting from the culturing of the strain, or a lysate thereof, and the strain itself may also be included in the culture product.

In an embodiment of the present invention, the lysate of the Akkermansia sp. strain may be a product obtained by lysing the strain itself either chemically or by applying physical force.

In an embodiment of the present invention, the extract may refer to a product obtained by extracting the strain itself, vesicles of the strain, a proteome and metabolome of the strain, a culture of the strain, a lysate of the strain, or a mixture thereof, and is used in a broad concept, including any substances that can be obtained by processing or treating the strain by other methods after the extraction. Specifically, the extract may be an ethanol fraction or an ethanol precipitate.

The pharmaceutical composition of the present invention may include a pharmaceutically acceptable carrier. The pharmaceutically acceptable carrier includes lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, and the like. In addition, it includes diluents or excipients such as fillers, extenders, binders, wetting agents, disintegrants, and surfactants.

The pharmaceutical composition may be formulated in the form of an oral formulation such as powder, granule, tablet, capsule, suspension, emulsion, syrup, or aerosol, a parenteral formulation, an injection formulation, and a topical administration formulation according to a conventional method.

Among oral formulations, the solid formulation for oral administration may include tablets, pills, powders, granules, capsules, and the like, and the solid formulation may include at least one excipient, for example starch, calcium carbonate, sucrose or lactose, and gelatin, and may include a lubricant such as magnesium stearate or talc. Among oral formulations, the liquid formulation for oral administration may include suspensions, solutions, emulsions, syrups, etc., and may include diluents such as water and liquid paraffin, wetting agents, sweeteners, fragrances, preservatives, or the like. The parenteral formulation includes sterilized aqueous solutions, non-aqueous solvents, suspensions, emulsions, and lyophilized formulations, and non-aqueous solvents and suspensions include propylene glycol, polyethylene glycol, vegetable oils such as olive oil, injectable esters such as ethyl oleate, and the like.

In addition, the pharmaceutical composition may be administered to mammals including or not including humans via various routes, for example, by means of oral administration, intravenous, intramuscular, or subcutaneous injection.

The composition of the present invention may be administered topically. The topical administration refers to direct application of active ingredients or compositions (such as skin external application compositions) to the skin and/or hair. The topical compositions of the present invention may be in the form of solutions, lotions, cerate, creams, ointments, liposomes, sprays, gels, foams, roller sticks, or any other formulation that is ordinarily used in dermatology.

In the pharmaceutical composition of the present invention, the dosage of the active ingredient may vary depending on the condition and body weight of a patient, the severity of the disease, the form of drug, and the route and duration of administration, but may be appropriately selected depending on the case. A preferred dosage of the pharmaceutical composition of the present invention may range from 0.001 mg/kg to 10 g/kg per day, preferably from 0.001 mg/kg to 1 g/kg, depending on the patient's condition, body weight, gender, age, severity, and the route of administration. Administration may be performed once or several times a day. Such a dosage should not be construed as limiting the scope of the present invention in any way.

Another aspect of the present invention relates to a food composition for preventing or inhibiting alopecia and promoting hair growth. The food composition of the present invention includes, as an active ingredient, an Akkermansia sp. strain, of the strain, a proteome and metabolome of the strain, a culture of the strain, a lysate of the strain, an extract of the strain, an extract of the culture, or an extract of the lysate and at least one physiologically acceptable excipient, diluent or carrier. The Akkermansia sp. strain may be viable (live), dormant, inactivated, pasteurized or killed or any combination thereof.

Any of the Akkermansia sp. strain may be used, and for example, an Akkermansia muciniphila strain may be used. In the present invention, a specific example of the Akkermansia sp. strain includes the Akkermansia muciniphila EB-AMDK19 strain, which are isolated and deposited by the present applicant.

The food composition of the present invention may be prepared as a health functional food such as a functional beverage, a health supplement food, or a special nutritional supplement food, and the form of the food may be a beverage such as tea, juice, carbonated beverage, or ion beverage, a processed milk such as milk or yogurt, a food product such as gum, rice cake, Korean traditional sweets, bread, snacks, or noodles, a health functional food product formulations such as powders, tablets, or capsules, and the like.

The food composition of the present invention may contain a sweetener, a flavoring agent, a physiologically active ingredient, a mineral, or the like in addition to the active ingredient.

The sweetener may be used in an amount that makes food taste moderately sweet, and may be natural or synthetic. Examples of the natural sweetener may include sugar sweeteners such as corn syrup solids, honey, sucrose, fructose, lactose, and maltose.

The flavoring agent may be used to improve the taste or flavor, and both natural and synthetic favoring agents may be used. It is preferable to use the natural flavoring agent. The natural flavoring agent may be used for nutritional supplementation in addition to flavor. Examples of the natural flavoring agent include those obtained from apple, lemon, mandarin, grape, strawberry, peach, green tea leaves, Polygonatum odoratum, bamboo leaves, cinnamon, chrysanthemum leaves, jasmine, or the like. In addition, other natural flavoring agents include those from ginseng (red ginseng), bamboo shoots, aloe vera, and ginkgo nuts. As the synthetic flavoring agent, esters, alcohols, aldehydes, terpenes, and the like may be used.

As the physiological active substance, catechins such as catechin, epicatechin, gallocatechin, and epigallocatechin, or vitamins such as retinols, ascorbic acid, tocopherol, calciferol, thiamine, and riboflavin may be used.

As the mineral, calcium, magnesium, chromium, cobalt, copper, fluorides, germanium, iodine, iron, lithium, magnesium, manganese, molybdenum, phosphorus, potassium, selenium, silicon, sodium, sulfur, vanadium, zinc, or the like may be used.

In addition, the food composition of the present invention may include a preservative, an emulsifier, an acidulant, a thickener, and the like, as necessary, in addition to the sweetener and the like. Such preservatives, emulsifiers, and the like are known in the art, and any of those known in the art may be used.

Still another aspect of the present invention may be a cosmetic composition, and the cosmetic composition may include the Akkermansia sp. strain, preferably an Akkermansia muciniphila strain. The cosmetic composition of the present invention includes, as an active ingredient, an Akkermansia muciniphila EB-AMDK19 strain (KCTC 13761BP), vesicles of the strain, a proteome and metabolome of the strain, a culture of the strain, a lysate of the strain, an extract of the strain, an extract of the culture, or an extract of the lysate and at least one cosmetically acceptable excipient, diluent or carrier. The Akkermansia sp. strain may be viable (live), dormant, inactivated, pasteurized or killed or any combination thereof.

The cosmetic composition of the present invention may be prepared in various forms according to a conventional cosmetic preparation method. Specifically, the cosmetic composition may be prepared in a formulation such as hair tonic, hair cream, hair lotion, hair shampoo, hair rinse, hair conditioner, hair spray, hair aerosol, pomade, powder, gel, hair pack, hair treatment, an eyebrow hair growth agent, an eyelash hair growth agent, an eyelash nutrition agent, or ointment.

In addition, the cosmetic composition may include general additives such as antioxidants, stabilizers, solubilizers, vitamins, pigments and flavors commonly used in the field of cosmetic compositions, and may include a carrier acceptable for cosmetics.

Examples of carriers acceptable for cosmetics include, but are not limited to, purified water, oil, wax, fatty acid, fatty acid alcohol, fatty acid ester, surfactant, humectant, thickener, viscosity stabilizer, chelating agent, buffering agent, preservative, lower alcohol, and the like. If desired, the carrier may include moisturizing agents, antiinflammatory agents, antibacterial agents, antifungal agents, vitamins, UV screening agents, antibiotics, perfumes, and dyes.

Taking the above results together, the composition for preventing or treating alopecia according to the present invention has not only excellent efficacy as an agent for preventing or treating alopecia, but also can particularly prevent, relieve, and combat scalp aging, thereby relieving and ameliorating alopecia which might be accelerated.

Hereinafter, the present invention will be described in detail with reference to examples. However, the following example is only illustration for assisting the overall understanding of the present invention, and the contents of the present invention are not limited by the following example.

### EXAMPLE

### Example 1

### 1.1 Strain Sample

The Akkermansia muciniphila EB-AMDK19 (KCTC 13761BP) live bacteria used in this experiment was prepared at a concentration of 1×10⁸ CFU/100 μL PBS (25% glycerol, 0.05% cysteine/PBS).

### 1.2. Animal Experiment

The animal experiment was carried out in compliance with the Animal use and Care Protocol of the Institutional Animal Care and Use Committee (IACUC). Seven-week-old male C57BL/6 mice were purchased and acclimated for 1 week, and then bred for 5 weeks. Regarding the breeding environment, the mice were bred at a constant temperature (22° C.) and relative humidity (40% to 60%) with a 12-hr light/12-hr dark cycle. A chow diet was fed to all experimental groups.

### 1.3 Sample Administration and Experimental Group Setting

As shown in Table 1 below, the mice were divided into four groups (8 mice/group), and each drug was subcutaneously injected and orally administrated to each mouse daily for 5 weeks 5 times a week. In order to make an alopecia model, 0.5 mg/day of testosterone was subcutaneously injected to mice in all experimental groups. A group in which alopecia was induced by testosterone administration was used as a negative control group, and finasteride was orally administered at 0.5 mg/kg/day as a positive control group. In the groups in which live bacteria and killed bacteria of the genus Akkermansia of the present invention are administrated, PBS containing L-cysteine and glycerol was orally administered as a vehicle.

TABLE 1

| Group | Experimental groups | Administrated drugs |
|---|---|---|
| I | Induced alopecia group | Testosterone 0.5 mg/day subcutaneous injection |
| | (Testosterone) | |
| II | Positive control group | Testosterone 0.5 mg/day subcutaneous injection |
| | (T + Finasteride) | Finasteride 0.5 mg/kg/day oral administration |
| III | EB-AMDK19 strain | Testosterone 0.5 mg/day subcutaneous injection |
| | administrated group | EB-AMDK19 live bacteria, 1 × 10⁸/CFU/100 μL |
| | (Live bacteria) | oral administration |
| | (T + EB-AMDK19) | |
| IV | EB-AMDK19 strain | Testosterone 0.5 mg/day subcutaneous injection |
| | administrated group | EB-AMDK19 killed bacteria, 1 × 10⁸ cells/100 μL |
| | (Killed bacteria) | oral administration |
| | (T + EB-AMDK19 HI) | |

### Test Example 1: Confirmation Experiment of Mouse Hair Growth Through Photograph

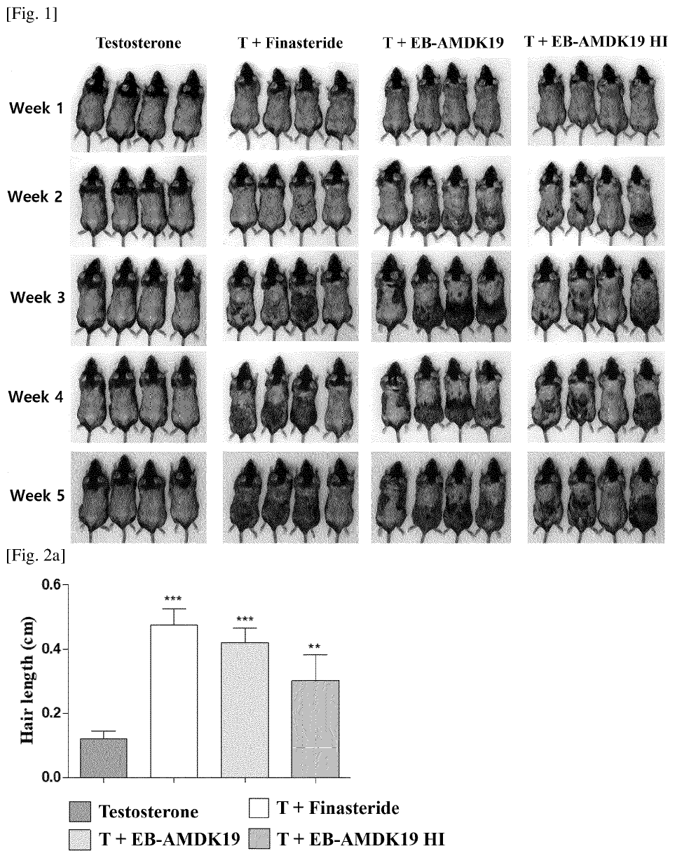

For the mice in each experimental group, the degree of hair growth was observed through photographing hair. FIG. 1 shows photographs showing the hair growth and regrowth of hair as the number of days has elapsed after each sample was applied to alopecia-induced mice. From the left side, there are the results of the negative control group (Testosterone), the positive control group (T+Finasteride), the Akkermansia muciniphila live bacteria-administered group (T+EB-AMDK19), and the Akkermansia muciniphila killed bacteria-administered group (T+EB-AMDK19 HI). By applying 100 μL of samples per day for 5 weeks to each group, it was checked whether hair growth occurred in hair loss regions of the backs of mice.

As shown in FIG. 1, the finasteride-treated group, which is a positive control group, showed a result that the skin color was darkened from week 3 of treatment, and the growth of hair shaft was expected, and as time passed, the range gradually expanded, and almost all regions thereof were changed to black at week 5.

In the negative control group (Testosterone), the skin color remained pink until week 5 when observation was completed, and it was found that hair did not grow in almost all regions.

In the mice in which the sample containing the Akkermansia muciniphila live bacteria was applied, a dark blue skin color was observed near the tail side of the hair loss regions after one week of the application, which means that the hair growth phenomenon is expected to occur faster than that of the positive control group. As a result, hair growth was evident in the lower part of the hair loss regions from week 3 after the application.

Meanwhile, in the case of mice in which the sample containing the Akkermansia muciniphila killed bacteria was applied, a dark blue skin color was partially observed in the center of the hair loss regions after one week of the application like the live bacteria-administered group, and hair growth was evident in the lower half region, in which hair loss occurred, from week 4 after the application.

### Test Example 2: Hair Growth Length Increase Effect

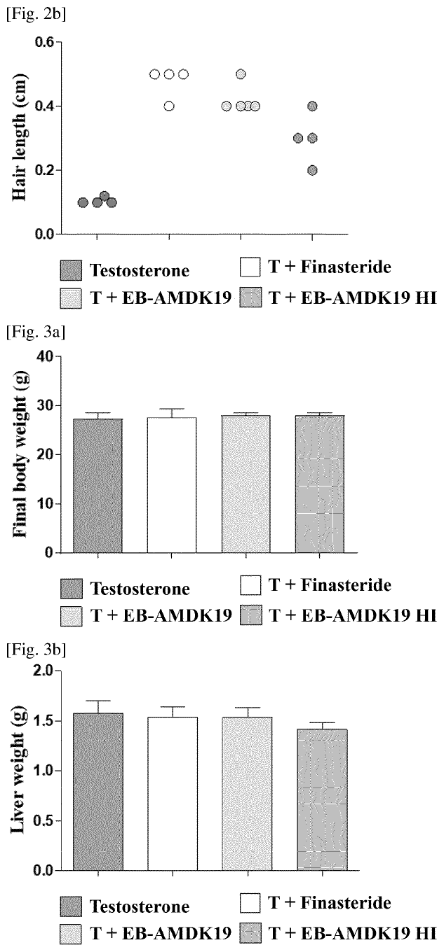

After the 5-week experiment was completed, the hair length was measured for individuals in each experimental group to calculate an average value, and the results of the hair growth length increase effect experiment are shown as a graph in FIG. 2.

Referring to FIGS. 2a-b, in the case of the negative control group to which distilled water was applied, it may be seen that there is no change such as the rapid growth of the hair length.

The hair lengths of the finasteride-administered group, which is a positive control group (T+Finasteride), and the live Akkermansia bacteria-administered group (T-EB-AMDK19) showed similar patterns, whereas the hair length of the killed Akkermansia bacteria-administered group (T+EB-AMDK19 HI) was relatively short.

As a result, as shown in FIGS. 2a-b, it was confirmed that the Akkermansia sp. strain was treated, and thus the hair length was significantly increased in the case of the live and killed bacteria treatment compared to the control group.

### Test Example 3: Measurement of Body Weight and Tissue Weight

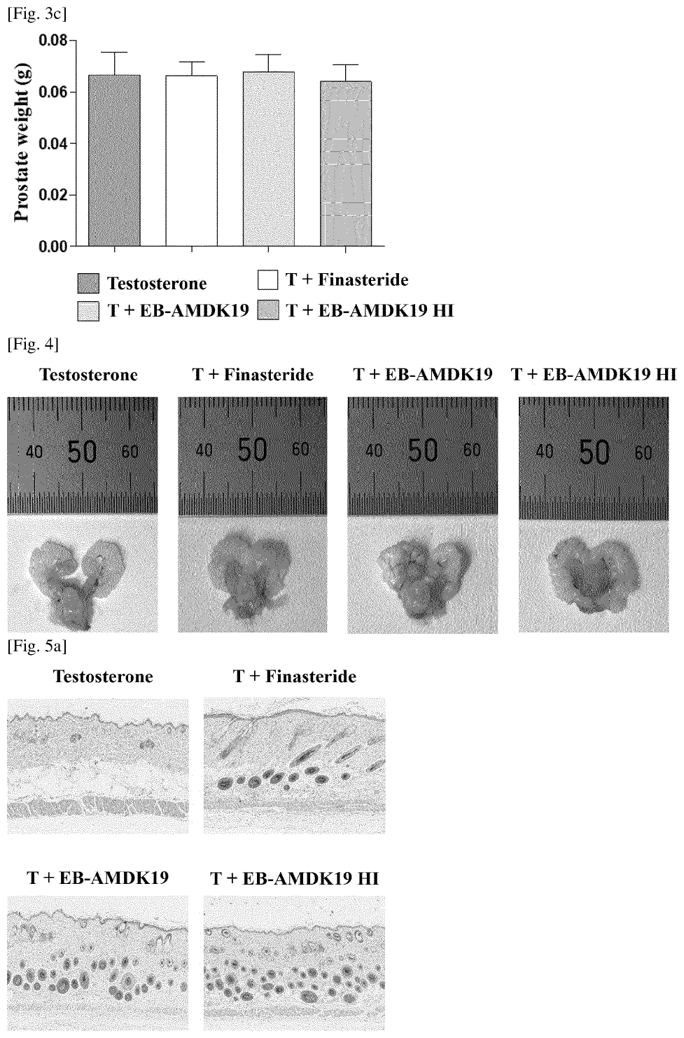

After the 5-week experiment was completed, mice in each group were sacrificed, liver and prostate tissues were removed, and the weights thereof were measured, and the results are shown in FIGS. 3a-c. The prostate was photographed and compared (see FIG. 4).

There was no change in body weight of the mice in each group, and there was no significant difference in the weight of the liver or prostate. There was no significant change in the size of the prostate.

### Test Example 4: Experiment of Measuring Histological Change in Mouse Hair Follicles

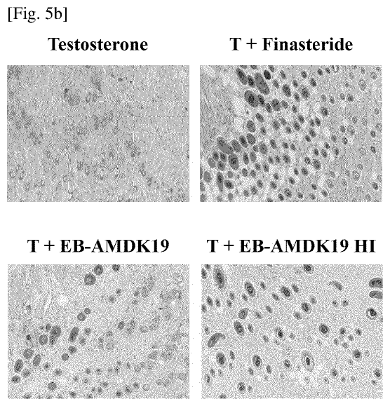

In order to confirm the morphological change of male C57BL/6 mouse hair follicles, skin tissues collected at week 5 after transdermal administration were stained with hematoxylin-eosin and observed with an optical microscope to confirm the number of hair follicles, and the results are shown in FIGS. 5a and 5b.

As a result of the measurement of the number of telogen hair follicles according to sample treatment in the alopecia-induced C57BL/6 mice, as shown in FIGS. 5a-b, it may be confirmed that when the mice were treated with finasteride, the number of the telogen hair follicles was increased compared to the negative control group.

In contrast, it may be seen that when the live Akkermansia sp. strain (live bacteria) of the present invention was administered, the number of telogen hair follicles significantly increased compared to the negative control group. In particular, it may be seen that the number of telogen hair follicles significantly increased when the killed Akkermansia sp. strain of the present invention was administered.

The above-described subject matter is to be considered illustrative, and not restrictive, so that it will be understood by those skilled in the art that various changes and modifications may be made therein without departing from the spirit and scope of the present disclosure. The true scope of protection of the present invention should be interpreted according to the claims, and all technical ideas that fall within the scope of equivalents thereof should be construed as being included in the scope of the present invention.

### [Accession Number]

Name of depositary institution: Korea Research Institute of Bioscience and Biotechnology
Accession No.: KCTC 13761BP
Date of Deposit: Dec. 5, 2018

1. A composition comprising, as an active ingredient, an Akkermansia sp. strain, vesicles of the strain, a proteome and metabolome of the strain, a culture of the strain, a lysate of the strain, an extract of the strain, an extract of the culture, or an extract of the lysate and at least one pharmaceutically acceptable excipient.

2. The composition of claim 1, wherein the Akkermansia sp. strain is an Akkermansia muciniphila strain.

3. The composition of claim 2, wherein the Akkermansia muciniphila strain is an Akkermansia muciniphila EB-AMDK19 (KCTC 13761BP).

4. The composition of claim 1, wherein the Akkermansia sp. strain is viable (live), dormant, inactivated, pasteurized or killed or any combination thereof.

5. The composition of claim 1, the composition is used for the treatment of one or more alopecia selected from the group consisting of androgenetic alopecia, telogen effluvium, alopecia areata, alopecia totalis, hypotrichosis, hereditary hypotrichosis simplex, frontal fibrosing alopecia, cicatricial alopecia, lichen planophilaris, ring alopecia, scarring alopecia, nonscarring alopecia, chemotherapy induced alopecia, and alopecia universalis.

6. The composition of claim 1, wherein the composition is a pharmaceutical composition and the pharmaceutical composition further comprises one or more therapeutic agent for alopecia selected from the group consisting of finasteride, dutasteride, minoxidil, episteride, alfatradiol, tofacitinib, and ruxolitinib.

7. (canceled)

8. (canceled)

9. (canceled)

10. The feed composition of claim 1, wherein the composition is a food composition and the food composition comprises 1×10⁴ CFU/mL to 1×10¹² CFU/mL of the Akkermansia sp. strain.

11. A cosmetic composition comprising, as an active ingredient, an Akkermansia sp. strain, vesicles of the strain, a proteome and metabolome of the strain, a culture of the strain, a lysate of the strain, an extract of the strain, an extract of the culture, or an extract of the lysate and at least one cosmetically acceptable excipient, diluent or carrier.

12. The cosmetic composition of claim 11, wherein the Akkermansia sp. strain is an Akkermansia muciniphila strain.

13. The cosmetic composition of claim 12, wherein the Akkermansia muciniphila strain is an Akkermansia muciniphila EB-AMDK19 strain (KCTC 13761BP).

14. The cosmetic composition of claim 11, which is formulated as a hair tonic, hair cream, hair lotion, hair shampoo, hair rinse, hair conditioner, hair spray, hair aerosol, pomade, powder, gel, hair pack, hair treatment, an eyebrow hair growth agent, an eyelash hair growth agent, an eyelash nutrition agent, or ointment.

15. A method for treating alopecia in a subject in need thereof, comprising administering to the subject a composition comprising, as an active ingredient, an Akkermansia sp. strain, vesicles of the strain, a proteome and metabolome of the strain, a culture of the strain, a lysate of the strain, an extract of the strain, an extract of the culture, or an extract of the lysate and at least one pharmaceutically acceptable excipient.

16. The method of claim 15, wherein the Akkermansia sp. strain is an Akkermansia muciniphila strain.

17. The method of claim 16, wherein the Akkermansia muciniphila strain is an Akkermansia muciniphila EB-AMDK19 (KCTC 13761BP).

18. The method of claim 15, wherein the Akkermansia sp. strain is viable (live), dormant, inactivated, pasteurized or killed or any combination thereof.

19. The method of claim 15, wherein the alopecia is selected from the group consisting of androgenetic alopecia, telogen effluvium, alopecia areata, alopecia totalis, hypotrichosis, hereditary hypotrichosis simplex, frontal fibrosing alopecia, cicatricial alopecia, lichen planophilaris, ring alopecia, scarring alopecia, nonscarring alopecia, chemotherapy induced alopecia, and alopecia universalis.

20. The method of claim 15, wherein the composition further comprises at least one therapeutic agent for alopecia selected from the group consisting of finasteride, dutasteride, minoxidil, episteride, alfatradiol, tofacitinib, and ruxolitinib.
